(19) United States
(12) Patent Application Publication
(10) Pub. No.: US 2009/0111747 A1
(43) Pub. Date: Apr. 30, 2009

(54) Anti-Diabetic or Anti-Hypertensive Dietary Supplement

(76) Inventors: Harry Stephen Ewart, Halifax (CA); Dorothy Anne Dennis, Halifax (CA); Colin Barrow, Halifax (CA)

Correspondence Address: SMART & BIGGAR;P.O. BOX 2999, STATION D, 900-55 METCALFE STREET, OTTAWA, ON, K1P 5Y6, CA

(21) Appl. No.: 11/883,720

(22) PCT Filed: Feb. 13, 2006
(86) PCT No.: PCT/CA06/00208

§ 371 Date: Jan. 22, 2008

Related U.S. Application Data
(60) Provisional application No. 60/651,977, filed on Feb. 14, 2005.

(51) Int. Cl. A61K 38/17; C12P 21/06

(52) U.S. Cl. 514/12; 435/68.1

(57) ABSTRACT

An anti-diabetic or anti-hypertensive fish protein hydrolysate is provided, in which the fish is of the genus Salmo or Oncorhynchus, and wherein the fish protein is hydrolysed by a metalloendopeptidase obtainable from Bacillus amyloliquefaciens. Methods of making and methods for using such fish protein hydrolysates are also provided.

### CROSS-REFERENCE TO RELATED APPLICATIONS

This application claims the benefit of U.S. Provisional Patent Application No. 60/651,977, filed Feb. 14, 2005, and which is incorporated herein by reference.

### FIELD OF THE INVENTION

The invention concerns an anti-diabetic or anti-hypertensive composition and, in particular but not limited thereto, an anti-type II diabetic composition, a method of producing such composition and a dietary supplement made by way of such a method.

### BACKGROUND OF THE INVENTION

Diabetes mellitus is a syndrome characterised by hyperglycaemia resulting from absolute or relative impairment in insulin secretion and/or insulin action. It is to be distinguished from gestational diabetes, which results in high blood glucose that develops at any time during pregnancy; and diabetes insipidus which is caused by the inability of the kidneys to conserve water, which leads to frequent urination and pronounced thirst.

Diabetes mellitus, commonly referred to as diabetes, means “sweet urine”.

There are two major types of diabetes mellitus: type I and type II diabetes. Type I diabetes mellitus is also called insulin dependent diabetes mellitus (IDDM), or juvenile onset diabetes mellitus.

In type I diabetes mellitus, the pancreas undergoes an autoimmune attack by the body itself, and is rendered incapable of making insulin. Abnormal antibodies have been found in patients with type I diabetes. Antibodies are proteins in the blood that are part of the body's immune system. The patient with type I diabetes must rely on insulin administration by, for example, injection for survival.

In type II diabetes [also referred to as non-insulin dependent diabetes mellitus or adult onset diabetes mellitus], patients can still produce insulin, but do so relatively inadequately. In many cases this actually means the pancreas produces larger than normal quantities of insulin. A major feature of type II diabetes is a lack of sensitivity to insulin by the cells of the body (particularly fat and muscle cells). In addition to the increase in insulin resistance, the release of insulin by the pancreas may also be defective, and occur late in response to increased glucose levels. Finally, the liver of patients with type II diabetes continues to produce glucose despite elevated glucose levels.

It is known that inhibition of α-glucosidase (an enzyme of the small intestine which catalyses the hydrolysis of terminal, non-reducing 1,4-linked α-D-glucose residues with release of D-glucose) is beneficial to patients with type II diabetes. Pharmaceutical preparations of α-glucosidase inhibitors are available from Bayer under the trade-marks Precose™ (acarbose) and Glyset® (miglitol). Acarbose is a complex oligosaccharide of microbial origin and miglitol is a desoxynojirimycin derivative.

The use of pharmaceutical α-glucosidase inhibitors is associated with gastrointestinal side effects, which has limited their use. The most common side effects are temporary digestive symptoms including abdominal discomfort, excessive gas (flatulence), and diarrhoea.

An alternative approach is to use milder, medicinal plant or food-based substances with α-glucosidase inhibitory properties.

Food-derived α-glucosidase inhibitors include Touchi extract, a fermented soybean product; Phase 2® (Pharmachem Labs), a water soluble extract of the white kidney bean Phaseolus vulgaris; and Salaretin® (Sami Labs), an extract from Salacia reticulata, a Sri-Lankan plant traditionally used in the ayurvedic system of Indian medicine.

Peptide inhibitors of α-glucosidase activity can be generated by enzymatic hydrolysis of food proteins (Matsui et al., 1996). However, the identity of active peptide inhibitors have been reported only from sardine and potato hydrolysates.

In sardine muscle protein hydrolysate at least part of the α-glucosidase inhibitory activity is due to the presence of the peptides Tyr-Tyr-Pro-Leu and Val-Trp (Matsui and Oki, 1999). The original in vitro assays were performed with α-glucosidase from brewer's yeast (Matsui et al., 1996) and subsequent work has shown that neither the hydrolysate nor the peptides had any significant inhibitory effect on mammalian α-glucosidase (Oki et al., 1999).

Five peptides with α-glucosidase inhibitory activity were isolated from a potato protein hydrolysate (Inoue et al., 2000). These peptides, Ile-Ile-Ser-Ile-Gly, Ile-Ile-Ser-Ile-Gly-Arg, Val-Phe-Ile-Lys-Ala-Ala, Val-Phe-Ile-Lys-Ala-Ala-Ala, Val-Phe-Ile-Lys-Ala are active against rat intestinal α-glucosidase.

### SUMMARY OF THE INVENTION

The present invention in a first aspect provides an anti-diabetic fish protein hydrolysate, in which said fish is of the genus Salmo or Oncorhynchus, and wherein the fish protein is hydrolysed by a metalloendopeptidase obtainable from Bacillus amyloliquefaciens.

In another aspect the present invention provides an anti-diabetic fish protein hydrolysate, in which said fish is of the genus Salmo or Oncorhynchus, and wherein the fish protein is hydrolysed by
a) a metalloendopeptidase obtainable from Bacillus stearothermophilus, followed by
b) a metalloendopeptidase obtainable from Bacillus amyloliquefaciens.

In another aspect, the present invention further provides an anti-diabetic composition comprising an anti-diabetic fish protein hydrolysate.

In still another aspect, the present invention further provides a dietary supplement, nutraceutical product, or functional food product comprising an anti-diabetic fish protein hydrolysate.

In yet another aspect, the present invention further provides a method for treating or preventing type II diabetes in a patient comprising administering to a patient in need thereof an anti-diabetic fish protein hydrolysate.

The invention further provides, in another aspect a method for inhibiting α-glucosidase activity in a patient comprising administering to a patient in need thereof an anti-diabetic fish protein hydrolysate.

In still another aspect, the present invention provides a method for reducing mean systolic blood pressure comprising administering to a patient in need thereof a fish protein hydrolysate of the invention.

In still yet another aspect, the present invention provides a method of producing an anti-diabetic dietary supplement comprising hydrolysing fish protein with a metalloendopeptidase obtainable from Bacillus amyloliquefaciens, wherein said fish is of the genus Salmo or Oncorhynchus.

In still yet another aspect, the present invention provides a method of producing an anti-diabetic dietary supplement comprising the steps of:
a) hydrolysing fish protein with a metalloendopeptidase obtainable from Bacillus stearothermophilus to form a hydrolysate,
b) isolating the aqueous insoluble phase of the hydrolysate, and
c) hydrolysing the aqueous insoluble phase of the hydrolysate with a metalloendopeptidase obtainable from Bacillus amyloliquefaciens,
   wherein said fish is of the genus Salmo or Oncorhynchus.

In still a further aspect, the present invention provides an anti-diabetic fish hydrolysate, obtained or obtainable by the method according to the present invention.

### BRIEF DESCRIPTION OF THE DRAWINGS

FIG. 1A depicts a flow chart of the hydrolysis of fish protein by a metalloendopeptidase such as Multifect® Neutral.

FIG. 1B depicts a flow chart of the hydrolysis of fish protein by a metalloendopeptidase such as Protease S Amano and hydrolysing the aqueous insoluble protein residue by a metalloendopeptidase with a broader specificity such as Multifect® Neutral.

FIGS. 2A and 2B depict graphs of the molecular weight profile of salmon protein hydrolysate prepared by Multifect® Neutral.

FIG. 3 depicts a graph of changes in systolic blood pressure after administration of salmon protein hydrolysate prepared by Multifect® Neutral to spontaneous hypertensive rats.

### DETAILED DESCRIPTION OF THE INVENTION

### Fish Species

The fish species used in accordance with the present invention may be of the genus Salmo or Oncorhynchus. Most preferably, the fish are selected from the group consisting of atlantic salmon (Salmo salar), coho salmon (Oncorhynchus kisutch), chinook salmon (Oncorhynchus tshawytscha) and steelhead salmon (Oncorhynchus mykiss), pink salmon (Oncorhynchus gorbuscha), and sockeye salmon (Oncorhynchus nerka).

The fish used in the invention may comprise the whole fish, a fillet, a rack, other fish parts, an extract or purified or partially purified fish proteins.

### Enzymes

Protease M Amano, Protease S Amano and Proleather FG-F may be obtained from Amano Enzyme USA (Lombard Ill., USA). Multifect® Neutral was obtained from Genencor International Inc. (Rochester, N.Y., USA).

Multifect® Neutral is a metalloendopeptidase obtainable from a controlled fermentation of a non-genetically modified strain of Bacillus amyloliquefaciens. Multifect® Neutral is characterised by its ability to hydrolyse a broad range of substrates at neutral pH.

Multifect® Neutral may be further characterised by its CAS (Chemical Abstract Services) number: 795297-30-2.

Protease S Amano is a metalloendopeptidase obtainable from Bacillus stearothermophilus fermentation. Protease S Amano catalyses the hydrolysis of peptide bonds on the C-terminal side of, in descending order with the most preferred amino acid first, arginine, alanine, lysine, phenylalanine and leucine.

Protease S Amano may be further characterised by its CAS (Chemical Abstract Services) number: 9001-92-7.

### Preparation of Hydrolysates

The fish fillet, rack or whole fish may be ground using a grinding machine known to those of skill in the art. The fish may also be de-boned using a de-boning apparatus prior to grinding.

The ground whole fish, fillet, rack; other fish parts, and extracts or fish proteins may be homogenized in water, for example, in a 1:1 ratio.

The water may contain an anti-bacterial agent such as methyl and/or propyl parabens to minimize bacterial degradation. Typically, if both methyl- and propylparabens are added it may be in the ratio, 2 parts methylparabens and 1 part propylparabens. Optionally, a further preservative may be added.

The protein present in the homogenate may be preferably denatured using, for example, heat prior to hydrolysis.

The denaturing temperature may be, for example, greater than 65° C., most preferably, about 70° C.

Preferably, the denaturing step may be from 5 to 20 minutes in duration. More preferably, the denaturing step may be from 5 to 15 minutes in duration. Most preferably, the denaturing step may be about 10 minutes in duration.

After denaturing, the mixture may preferably be cooled to 50° C. and the pH of the mixture adjusted to, for example, between about pH 6 to pH 9 by the drop wise addition of 1N sodium hydroxide.

Advantageously, the provision of an anti-hypertensive and anti-diabetic composition and dietary supplement is achievable by a single enzymatic digest or successive enzymatic digests.

The protein content in the fish may be determined by a method known to those skilled in the art, for example, by the Kjeldahl nitrogen method wherein the percentage protein is equal to the percentage nitrogen multiplied by 6.25. The degree of hydrolysis may be determined by the OPA reaction method.

### Single Enzymatic Digest

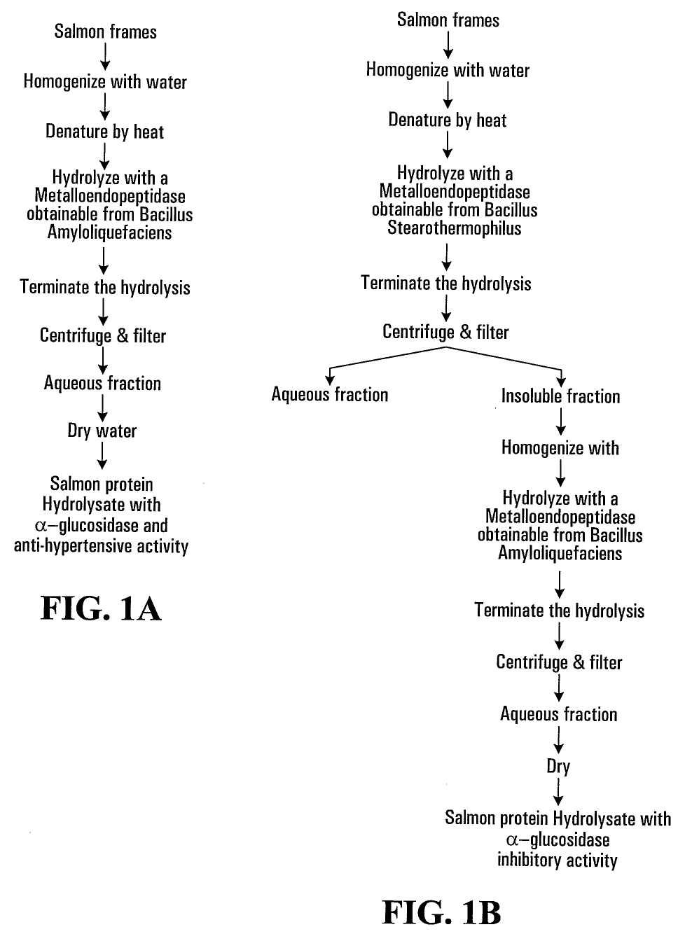

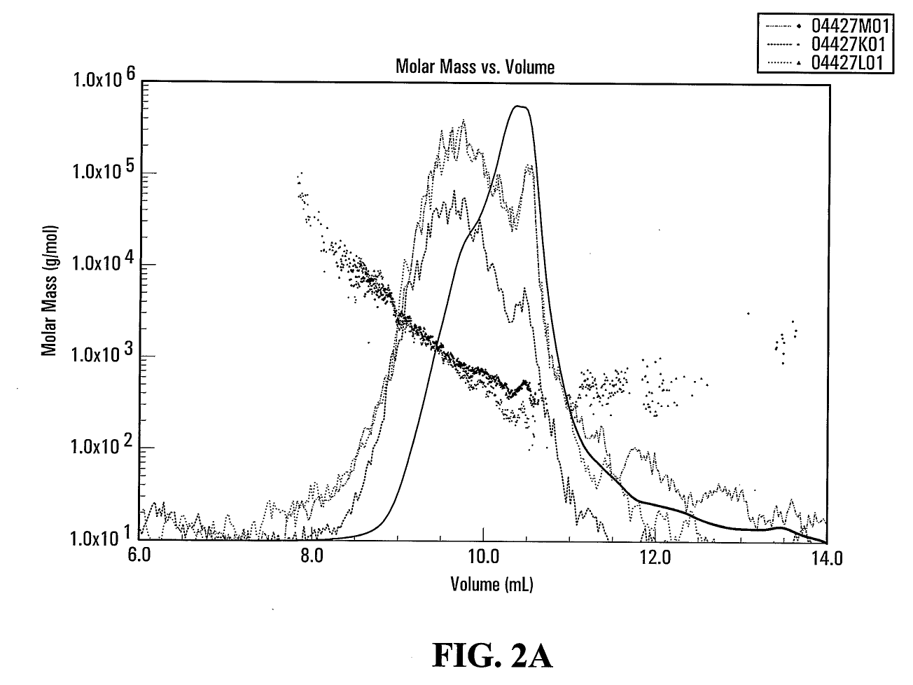

A single enzymatic hydrolysis may be carried out using a metalloendopeptidase obtainable from Bacillus amyloliquefaciens such as, for example, a bacterial neutral protease available from Genencor International Inc. under the trade-mark Multifect® Neutral. A single enzymatic digest is outlined in FIG. 1A.

When a digest is performed with a metalloendopeptidase obtainable from Bacillus amyloliquefaciens, preferably the pH is adjusted to about 7, and the protease may be added at a ratio of from about 1.0% to 12% w/w metalloendopeptidase to fish protein substrate. For example, the ratio may be from about 8% to 11%, for example, about 10%.

Hydrolysis of the fish protein may be performed at a temperature of from 10° C. to 60° C. Preferably the hydrolysis is carried out at a temperature of from 45° C. to 55° C., most preferably at a temperature of about 50° C.

Preferably, a degree of hydrolysis of about 10% to 60% may be achieved. More preferably, a degree of hydrolysis of about 25 to 35% may be achieved. Most preferably, a degree of hydrolysis of between 25 to 35%, for example, 28 to 30%.

Typically this takes from 1 to 24 hours to achieve.

Preferably, the hydrolysis reaction proceeds for 4.5 to 5.5 hours, for example, at least 5 hours.

It is not necessary to constantly maintain a steady pH value of the homogenized fish mixture during the hydrolysis reaction. Heating the homogenized mixture to greater than 80° C. for longer than 3 minutes to inactivate the metalloendpeptidase stops the hydrolysis reaction.

The resulting hydrolysate comprises an aqueous soluble phase and aqueous insoluble phases. The insoluble phases may be removed by, for example, centrifugation and filtration.

The aqueous soluble phase is retained and dried such as by spray drying to obtain a powdered fish protein hydrolysate.

Alternatively, the aqueous soluble phase may be concentrated using a rotary evaporator and then lyophilized or spray dried, to yield a concentrated, powdered, protein hydrolysate.

The aqueous soluble phase may be further processed either before or after drying by for example, ethanol precipitation or ultrafiltration to remove high molecular weight peptides or protein fragments. The aqueous soluble phase may also be further processed by, for example, filtration, chromatography, dialysis and/or centrifugation, or any combination thereof, as are known in the art.

Enzymatic Digest with Two Enzymes

Alternatively, enzymatic hydrolysis may be carried out using successive digestions using a metalloendopeptidase obtainable from Bacillus stearothermophilus and hydrolysing the aqueous insoluble protein by a metalloendopeptidase obtainable from Bacillus amyloliquefaciens. A metalloendopeptidase obtainable from Bacillus stearothermophilus is available under the trade-mark Protease S Amano from Amano Enzyme U.S.A. The aqueous phase of this digest may be removed and the remaining insoluble phase may then be digested with a metalloendopeptidase obtainable from Bacillus amyloliquefaciens such as, for example, a bacterial neutral protease available under the trade-mark Multifect® Neutral from Genencor International Inc. An enzymatic digest with two enzymes in succession is outlined in FIG. 1B.

When hydrolysis is performed with a metalloendopeptidase obtainable from Bacillus stearothermophilus followed by one obtainable from Bacillus amyloliquefaciens, the pH of the homogenate, may be adjusted to an initial value of about 8.0, the optimal pH for the metalloendopeptidase, by the addition of 1N NaOH.

The metalloendopeptidase may be added at, for example, about 1 to 5%, for example about 3% w/w enzyme/substrate (protein in the fish racks) ratio and hydrolysis carried out at about 50° C. for approximately 7 hours until a degree of hydrolysis of about 10 to 30% is achieved. A degree of hydrolysis of about 17% is particularly preferred. The pH need not be maintained at a constant value. The mixture may be heated to about 85° C. and held there for about 10 minutes to terminate the hydrolysis by inactivating the enzyme.

After the initial digest with the metalloendopeptidase obtainable from Bacillus stearothermophilus, the resulting hydrolysate comprises an aqueous soluble phase and an aqueous insoluble phase. The aqueous insoluble phase may be recovered by, for example, centrifugation. The aqueous insoluble phase may contain insoluble protein. The aqueous insoluble phase may then be subjected to a further round of hydrolysis with a metalloendopeptidase obtainable from Bacillus amyloliquefaciens.

The aqueous insoluble phase are homogenized in, for example, water in about a 1:1 ratio. The pH of the mixture may be adjusted to about 7.0, the optimal pH of the metalloendopeptidase, by the addition of 1N NaOH. The metalloendopeptidase may be added at a ratio of about 8% w/w enzyme/substrate (protein in the initial fish racks). The hydrolysis reaction may be carried out at about 50° C. for about 5 hours until a degree of hydrolysis of about 10 to 60% is achieved. A degree of hydrolysis of about 30% is particularly preferred. The pH need not be maintained at a constant value. The mixture may then be heated to about 85° C. for about 10 minutes to terminate the hydrolysis reaction.

The final hydrolysate comprises an aqueous soluble phase and an aqueous insoluble phase.

The aqueous insoluble phase may be removed by, for example, centrifugation and vacuum filtration through a suitable filter or membrane, for example, diatomaceous earth.

The aqueous soluble phase is retained and dried such as by spray drying to obtain a powdered fish protein hydrolysate.

Alternatively, the aqueous soluble phase may be concentrated with a rotary evaporator and then lyophilized or spray dried to yield a concentrated, powdered, protein hydrolysate.

The aqueous soluble phase may be further processed either before or after drying by, for example, ethanol precipitation or ultrafiltration to remove high molecular weight peptides or protein fragments. The aqueous fraction may also be further processed by, for example, filtration, chromatography, dialysis and/or centrifugation, or any combination thereof, as are known in the art.

### Uses

The fish protein hydrolysate of the present invention possesses useful anti-diabetic or anti-hypertensive properties. In particular the hydrolysate of the present invention may possess useful anti-type II diabetic properties.

The fish protein hydrolysate of the present invention may also be useful in the treatment, prevention and amelioration of the complications of type II diabetes. Typically, these include for example, diabetic retinopathy, diabetic nephropathy, diabetic neuropathy, peripheral vascular disease, high cholesterol, high blood pressure, atherosclerosis and coronary heart disease.

The use of the hydrolysate in accordance with the invention may also reduce mean systolic blood pressure in patients with type II diabetes by inhibiting angiotensin converting enzyme.

Hydrolysates in accordance with the present invention may also be useful in the treatment or prevention of obesity.

### Compositions

Compounds and compositions according to the present invention may be used in a variety of products, for example, pharmaceutical or nutraceutical products, dietary supplements, food products, food ingredients and beverages. The fish protein hydrolysate may be microencapulated in order to improve palatability or processing characteristics of the food or beverage products. Alternatively, the fish protein hydrolysates may be used on their own.

Preferably, formulations of compositions and compounds in accordance with the present invention are intended for oral administration. The formulations comprise the composition of the present invention in combination with one or more physiologically acceptable ingredients, such as carriers, excipients or diluents. Compositions and formulations for oral administration are particularly preferred. Formulations may be prepared, for example, in unit dose forms, such as tablets, capsules, sachets, dragees, or ampoules. They may be prepared in a conventional manner, for example by means of conventional mixing, granulating, confectioning, dissolving or lyophilizing processes.

Typical physiologically acceptable ingredients include:
a) binding agents such as starch, polyvinylpyrrolidone, hydroxyproylmethylcellulose and/or gelatine;
b) fillers such as sugars (e.g. lactose, saccharose, mannitol, sorbitol) and amylopectin, cellulose preparations (e.g. microcrystalline cellulose), calcium phosphates (e.g. tricalcium phosphate, calcium hydrogen phosphate, lactose) and/or titanium dioxide;
c) lubricants such as steric acid, calcium stearate, magnesium stearate, talc, silica, silicic acid, polyethylene glycol and/or waxes;
d) disintegrants such the above mentioned starches, carboxymethyl starch, cross-linked polyvinylpyrrolidone, agar, algenic acid or a salt thereof (e.g. sodium alginate) and/or sodium starch glycolate;
e) wetting agents such as sodium lauryl sulphate; and/or
f) stabilizers.

Soft gelatine capsules may be prepared with capsules containing a mixture of the fish protein hydrolysate composition together with paraffin oil, liquid polyethylene glycols, vegetable oil, fat and/or another suitable vehicle for soft gelatine capsules. Plasticisers such as glycerol or sorbitol may also be used. Hard gelatine capsules may contain granules of the composition. Hard gelatine capsules may also contain the composition in combination with solid powdered ingredients such as those listed above.

Liquid formulations for oral administration may be prepared in the form of solutions, syrups or suspensions. Liquid formulations typically comprise the fish protein hydrolysate composition with an excipient such as sugar or sugar alcohols, and a carrier such as ethanol, water, glycerol, propylene glycol, polyethylene glycol, almond oil, oily esters or mixtures thereof. If desired, such liquid formulations may also contain colouring agents, flavouring agents, saccharine, thickening agents (e.g. carboxy methyl cellulose), suspending agents (e.g. sorbitol syrup, methylcellulose, hydrogenated edible fats), emulsifying agents (e.g. lecithin, acacia), and/or preservatives (e.g. methyl p-hydroxy benzoates, propyl p-hydroxy benzoates, sorbic acid). Liquid formulations for oral administration may also be prepared in the form of a dry powder to be reconstituted with water or another suitable vehicle prior to use.

Formulations may contain one or more additional active ingredients, particularly one or more further anti-diabetic agents. The one or more further anti-diabetic agents is preferably selected from the group consisting of sulphonylureas, for example, chlorpropamide, tolbutamide, glyburide, glipizide and glimepiride; meglitinides, for example, repaglinide and nateglinide; biguanides, for example, metformin; thiazolidinediones, for example, pioglitazone and rosiglitazone.

High blood pressure is a complication of diabetes mellitus. Accordingly, the one or more additional active ingredients may be anti-hypertensive agents useful in the control of high blood pressure. Typically these agents may be selected from the group consisting of angiotensin converting enzyme inhibitors, for example, ramipril, lisinopril, captopril, enalapril and trandolapril.

An effective amount of the fish protein hydrolysate composition can be determined by the skilled person and may depend on various factors, such as the nature of the product, the condition to be prevented or treated, the method of administration, species of animal, age and/or individual condition. For example, the effective amount may be from 0.1 g to 5 g of the dried hydrolysate per day, for example, from 0.5 g to 3 g per day. The effective amount may be from 0.5 g to 2 g of the dried hydrolysate per day, for example, 1 g per day. The effective amount of the dried hydrolysate may be given between 1 to 4 times a day, for example, between 1 to 2 times a day.

The following examples are offered by way of illustration and not by way of limitation.

### EXAMPLES

### General Methods

### Substrates

The fish substrate used in these examples is a rack of atlantic salmon. The salmon was obtained from a fish processing plant (Heritage Salmon Limited, Blacks Harbour, New Brunswick, Canada).

### Determination of Protein Content

The protein content of the salmon racks was measured using the Kjeldahl nitrogen method wherein:

% protein=% nitrogen×6.25.

### Preparation of the Hydrolysates

FIGS. 1a and 1b provide a schematic outline of a typical protocol for providing hydrolysates in accordance with the present invention.

### Example 1

This example describes the production of salmon protein hydrolysates by individual proteolytic enzymes and the α-glucosidase inhibitory activity of the hydrolysates.

Ground salmon racks were homogenized in water at a 1:1 ratio. 0.013% parabens was added to the homogenate to minimise bacterial degradation.

The homogenate was then heated to 70° C. for 10 minutes to denature the salmon protein. The homogenate was then cooled to 50° C.

The homogenate was hydrolysed using Protease M Amano, Protease S Amano, Proleather FGF or Multifect® Neutral and the in vitro α-glucosidase activity of the hydrolysates were measured.

As can be seen in table 1, the pH of the homogenate was adjusted to an optimum pH for each respective enzyme using 1N NaOH. Proteases were then added to aliquots of the homogenate in the enzyme/substrate (E/S) ratio specified for the respective enzyme in Table 1. Hydrolysis was performed for 5 hours at 50° C.

The pH was not maintained at a constant value.

The homogenates were heated to 85° C. for 10 minutes to terminate the hydrolysis reaction.

The insoluble fraction was removed by centrifugation. The emulsion fraction and oil were removed by vacuum filtration through diatomaceous earth. The filtered aqueous fraction was spray dried to obtain a powdered salmon protein hydrolysate. The percentage αglucosidase inhibition of each hydrolysate was then determined (Table 1). The α-glucosidase inhibitory activity of a commercially available fish protein hydrolysate (dried bonito hydrolysed with thermolysin) was also measured. Touchi extract was measured as a positive control. The α-glucosidase inhibitory activity was measured by the modified method of Matsui et al. α-Glucosidase from rat intestinal acetone powder was used in the assay.

TABLE 1
The % inhibition of α-glucosidase for each hydrolysate is shown.

| | E/S | | Temperature | Time | % inhibition at |
|---|---|---|---|---|---|
| Protease | Ratio | PH | ° C. | (hrs) | 1.4 mg/ml |
| Protease M | 2.6 | 4.5 | 50 | 5 | 0 |
| Amano | | | | | |
| Protease S | 2.6 | 8.0 | 50 | 5 | 0 |
| Amano | | | | | |
| Proleather | 2.6 | 10.0 | 50 | 5 | 0 |
| FGF | | | | | |
| Multifect ® | 10.3 | 7.0 | 50 | 5 | 29.7 |
| Neutral | | | | | |
| Bonito | — | — | — | — | 0 |
| peptide | | | | | |
| Touchi | — | — | — | — | 53.3 |
| extract | | | | | |

As can be seen from Table 1, of the hydrolysates produced, the hydrolysate obtained using Multifect® Neutral demonstrated the most potent inhibitory activity.

### Example 2

This example describes production of a salmon protein hydrolysate by digestion with Protease S Amano followed by Multifect® Neutral and the α-glucosidase inhibitory activity of the hydrolysate.

50 g of ground salmon frames were homogenized with 50 g of water. 0.013% parabens was added to the homogenate to minimise bacterial degradation.

The homogenate was heated to 70° C. for 10 minutes to denature the salmon protein. The homogenate was then cooled to 50° C. and the pH of the homogenate was adjusted to 8.0, the optimal pH of Protease S Amano, by the addition of 1N NaOH. Protease S Amano was added at a ratio of 2.6% w/w enzyme/substrate (protein in the salmon frames, as determined by the Kjeldahl method above). The hydrolysis reaction was carried out at 50° C. for 7 h. The pH was not maintained at a constant value. The mixture was heated to 85° C. for 10 minutes to terminate the hydrolysis reaction.

The resulting hydrolysate comprised an aqueous soluble phase and aqueous insoluble phases. The aqueous insoluble phases were recovered by centrifugation.

After hydrolysis with Protease S Amano, a substantial amount of protein remains in the aqueous insoluble phases. The aqueous insoluble phases were homogenized in water in a 1:1 ratio. The homogenate was heated to 50° C. and the pH of the mixture was adjusted to 7.0, the optimal pH of Multifect® Neutral, by the addition of 1N NaOH. Multifect® Neutral was added at a ratio of 8% enzyme/substrate (protein in the salmon frames as determined by the Kjeldahl method above). The hydrolysis reaction was carried out at 50° C. for 5 h. The pH was not maintained at a constant value. The mixture was then heated to 85° C. for 10 minutes to terminate the hydrolysis reaction.

The resulting hydrolysate comprised an insoluble fraction, an emulsion and oil fraction and an aqueous fraction. The insoluble fraction was removed by centrifugation. The emulsion and oil fraction was removed by vacuum filtration through diatomaceous earth. The filtered aqueous fraction was freeze dried to obtain a powdered salmon protein hydrolysate. The α-glucosidase inhibitory activity (IC₅₀) was then determined (Table 2) by the method described in Example 1.

TABLE 2
The IC₅₀ of hydrolysates obtained from individual and combined
digests using Protease S Amano and Multifect ® Neutral are shown.

| | % inhibition at | |
|---|---|---|
| Protease | 0.8 mg/ml | IC₅₀ (mg/ml) |
| Protease S Amano | 0 | — |
| Multifect ® Neutral | 18.6 | 5.4 |
| Protease S Amano | 40.3 | 1.5 |
| followed by | | |
| Multifect ® Neutral | | |
| Touchi extract | 30.8 | 1.9 |

The hydrolysate, obtained by digestion with Protease S Amano followed by Multifect® Neutral demonstrates an inhibitory activity of α-glucosidase more potent than the hydrolysate obtained by either metalloendopeptidase individually and Touchi extract.

### Example 3

In this example the physiochemical properties of the salmon protein hydrolysate obtained by using Multifect® Neutral was analysed.

The degree of hydrolysis of the hydrolysate was assessed by the OPA method. The degree of hydrolysis was 29.9%.

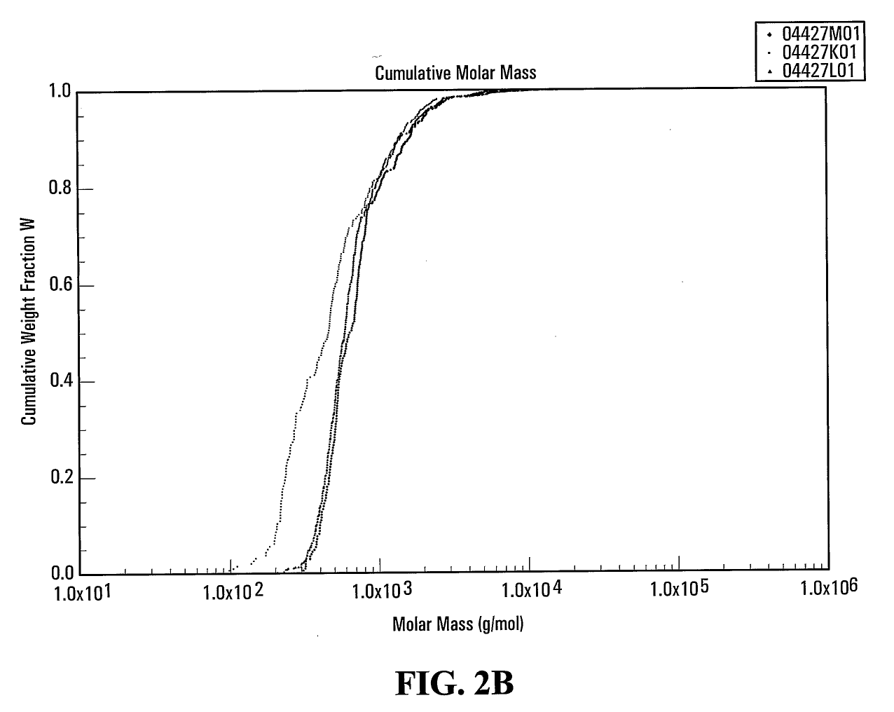

The hydrolysate powder was dissolved in 88 mM sodium acetate and loaded on to a TSK G3000PWXL gel filtration column. The column was eluted with 88 mM sodium acetate at a flow rate of 0.75 ml/min. The elution was monitored by multi-angle light scattering (MALS) and RI detection. FIG. 2a and FIG. 2b show that 94.8% of the peptides within the salmon protein hydrolysate obtained with a digest using Multifect® Neutral are less than 2000 daltons.

### Example 4

This example describes the reduction of systolic blood pressure by a salmon protein hydrolysate obtained using Multifect® Neutral alone (i.e. by the process depicted in FIG. 1A).

Blood pressure was measured in conscious rats via the tail cuff method. Male spontaneously hypertensive rats (10 weeks old) were acclimatized for 10-14 days after arrival with daily application of tail-cuff to the animals so as to accustom them to the experimental manipulations. On the day of the experiment, animals received by oral gavage, salmon hydrolysate dissolved in water at a dose of either 1500 or 3000 mg/kg body weight. Control animals received water only.

Systolic blood pressure measurements were taken just prior to dosing (time 0) and 1, 2, 4, 6 and 8 hours after. The cuff pressure and the pulsations were captured simultaneously with a data acquisition/monitoring device (Biopac Data Acquisition system).

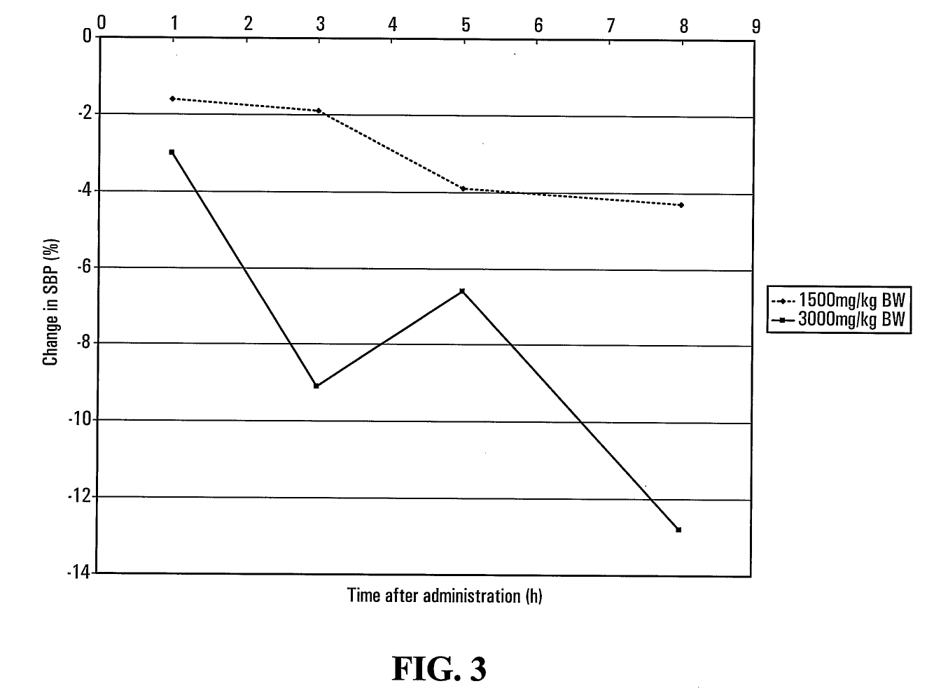

Referring to FIG. 3 it can be seen that the salmon protein hydrolysate prepared by hydrolysing salmon protein with Multifect® Neutral also reduces mean systolic blood pressure.

A single oral dose at 1500 mg/kg of body weight of the protein hydrolysate failed to produce a significant reduction in mean systolic blood pressure. However, an oral dose of 3000 mg/kg of body weight significantly reduced mean systolic blood pressure from −9.1% to −12.8% during the period 3 to 8 hours after administration.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

### REFERENCES

Inoue, A., Doge, T., Mitsuo, O., Shiomi, T. and Onodera, S α-Glucosidase inhibitor and its mixture JP10292000.
Matsui, T., Ueda, T., Oki, T., Sugita, K., Terahara, N., Matsumoto, K. (2001) α-Glucosidase inhibitory action of natural acylated anthocyanins. Survey of natural pigments with potent inhibitory activity. J. Agric. Food Chem. 49: 1948-1951.
Matsui, T. and Oki, T. (1999) Isolation and identification of peptidic α-glucosidase inhibitors derived from sardine muscle hydrolysate. Z. Naturforsch. 54C:4948-4952.
Matsui, T., Yoshimoto, C., Osajima, K., Oki, T. and Osajima, Y. (1996) In vitro survey of α-glucosidase inhibitory food components. Biosci. Biotech. Biochem. 60:2019-2022.
Oki, T., Matsui, T. and Osajima, Y. (1999) Inhibitory effect of α-glucosidase inhibitors varies according to its origin. J. Agric. Food Chem. 47: 550-553.

1. An anti-diabetic or anti-hypertensive fish protein hydrolysate, in which said fish is of the genus Salmo or Oncorhynchus, and wherein the fish protein is hydrolysed by a metalloendopeptidase obtained or obtainable from Bacillus amyloliquefaciens.

2. An anti-diabetic or anti-hypertensive fish protein hydrolysate, in which said fish is of the genus Salmo or Oncorhynchus, and wherein the fish protein is hydrolysed by
a) a metalloendopeptidase obtained or obtainable from Bacillus stearothermophilus, followed by
b) a metalloendopeptidase obtained or obtainable from Bacillus amyloliquefaciens.

3. The anti-diabetic or anti-hypertensive fish protein hydrolysate according to claim 1, wherein the fish is selected from the group consisting of atlantic salmon (Salmo salar), coho salmon (Oncorhynchus kisutch), chinook salmon (Oncorhynchus tshawytscha) and steelhead salmon (Oncorhynchus mykiss), pink salmon (Oncorhynchus gorbuscha), and sockeye salmon (Oncorhynchus nerka).

4. The anti-diabetic or anti-hypertensive fish protein hydrolysate according to claim 2, wherein the fish is selected from the group consisting of atlantic salmon (Salmo salar), coho salmon (Oncorhynchus kisutch), chinook salmon (Oncorhynchus tshawytscha) and steelhead salmon (Oncorhynchus mykiss), pink salmon (Oncorhynchus gorbuscha), and sockeye salmon (Oncorhynchus nerka).

5. (canceled)

6. The anti-diabetic or anti-hypertensive fish protein hydrolysate according to claim 2, wherein the metalloendopeptidase obtained or obtainable from Bacillus stearothermophilus is Protease S Amano.

7. The anti-diabetic or anti-hypertensive fish protein hydrolysate according to claim 1, wherein the metalloendopeptidase obtained or obtainable from Bacillus amyloliquefaciens is Multifect® Neutral.

8. An anti-diabetic or anti-hypertensive composition, dietary supplement, nutraceutical product, or functional food product comprising an anti-diabetic or anti-hypertensive fish protein hydrolysate according to claim 1.

9. An anti-diabetic or anti-hypertensive composition, dietary supplement, nutraceutical product, or functional food product comprising an anti-diabetic or anti-hypertensive fish protein hydrolysate according to claim 2.

10. (canceled)

11. (canceled)

12. A method comprising administering to a patient in need thereof an anti-diabetic or anti-hypertensive fish protein hydrolysate according to claim 1, said method being a method for treating or preventing diabetes in said patient, a method for inhibiting α-glucosidase enzyme activity in said patient, or a method for reducing mean systolic blood pressure in said patient.

13. A method comprising administering to a patient in need thereof an anti-diabetic or anti-hypertensive fish protein hydrolysate according to claim 2, said method being a method for treating or preventing diabetes in said patient, a method for inhibiting α-glucosidase enzyme activity in said patient, or a method for reducing mean systolic blood pressure in said patient.

14. (canceled)

15. A method for producing an anti-diabetic or anti-hypertensive fish protein hydrolysate according to claim 1, said method comprising hydrolysing fish protein with a metalloendopeptidase obtainable from Bacillus amyloliquefaciens, wherein said fish is of the genus Salmo or Oncorhynchus.

16. A method for producing an anti-diabetic or anti-hypertensive fish protein hydrolysate according to claim 2, comprising the steps of:
a) hydrolysing fish protein with a metalloendopeptidase obtainable from Bacillus stearothermophilus to form a hydrolysate,
b) isolating the aqueous insoluble phase of the hydrolysate, and,
c) hydrolysing the aqueous insoluble phase of the hydrolysate with a metalloendopeptidase obtainable from Bacillus amyloliquefaciens,
wherein said fish is of the genus Salmo or Oncorhynchus.

17. The method according to claim 15, wherein the fish is selected from the group consisting of atlantic salmon (Salmo salar), Coho salmon (Oncorhynchus kisutch), chinook salmon (Oncorhynchus tshawytscha) and steelhead salmon (Oncorhynchus mykiss), pink salmon (Oncorhynchus gorbuscha), and sockeye salmon (Oncorhynchus nerka).

18. The method according to claim 16, wherein the fish is selected from the group consisting of atlantic salmon (Salmo salar), Coho salmon (Oncorhynchus kisutch), chinook salmon (Oncorhynchus tshawytscha) and steelhead salmon (Oncorhynchus mykiss), pink salmon (Oncorhynchus gorbuscha), and sockeye salmon (Oncorhynchus nerka).

19. (canceled)

20. (canceled)

21. The method according to claim 15, comprising the step of denaturing the fish protein using heat prior to hydrolysis with the metalloendopeptidase obtainable from Bacillus amyloliquefaciens.

22. The method according to claim 16, comprising the step of denaturing the fish protein using heat prior to hydrolysis with the metalloendopeptidase obtainable from Bacillus stearothermophilus or Bacillus amyloliquefaciens.

23. (canceled)

24. (canceled)

25. (canceled)

26. (canceled)

27. (canceled)

28. An anti-diabetic or anti-hypertensive fish protein hydrolysate, obtained or obtainable by the method according to claim 15.

29. An anti-diabetic fish or anti-hypertensive fish protein hydrolysate, obtained by or obtainable the method according to claim 16.
